(19) United States
(12) Patent Application Publication
(10) Pub. No.: US 2006/0223135 A1
(43) Pub. Date: Oct. 5, 2006

(54) High throughput reactive oxygen species-based cytochrome P450 inhibition assay

(75) Inventors: Dhiren R. Thakker, Raleigh, NC (US); John H. Ansede, Cary, NC (US)

Correspondence Address: JENKINS, WILSON, TAYLOR & HUNT, P. A., 3100 TOWER BLVD, SUITE 1200, DURHAM, NC, 27707, US

(73) Assignee: The University of North Carolina at Chapel Hill

(21) Appl. No.: 11/099,427

(22) Filed: Apr. 5, 2005

(51) Int. Cl. C12Q 1/26

(52) U.S. Cl. 435/25

(57) ABSTRACT

A method of screening a candidate compound for an ability to inhibit a cytochrome P450. The method includes the steps of reacting the candidate compound, an indicator compound precursor, a cytochrome P450 substrate, and the cytochrome P450, the cytochrome P450 characterized as having a side reaction associated with metabolic activity of the cytochrome P450 wherein a chemical species capable of reacting with the indicator compound precursor is produced; quantifying the amount of indicator compound produced in the presence of the candidate compound, and comparing the amount of indicator compound produced in the presence of the candidate compound to an amount of indicator produced under identical conditions in the absence of the candidate compound, the comparison indicating the ability of the candidate compound to inhibit the cytochrome P450. Also disclosed is a method for determining an inhibitory potency of a candidate compound for a cytochrome P450.

### CROSS REFERENCE TO RELATED APPLICATIONS

This application is based on and claims priority to U.S. Provisional Application Ser. No. 60/559,570, filed Apr. 5, 2004, herein incorporated by reference in its entirety.

### TECHNICAL FIELD

The presently disclosed subject matter relates to a method of screening candidate compounds for an ability to inhibit a cytochrome P450. More particularly, the presently disclosed subject matter relates to a method of determining an inhibitory potency of such candidate compounds for cytochrome P450.

### Table of Abbreviations

CYP—cytochrome P450
CYP1A2—cytochrome P450 1A2
CYP2B6—cytochrome P450 2B6
CYP2C8—cytochrome P450 2C8
CYP2C9—cytochrome P450 2C9
CYP2C19—cytochrome P450 2C19
CYP2D6—cytochrome P450 2D6
CYP2E1—cytochrome P450 2E1
CYP3A4—cytochrome P450 3A4
CYP3A5—cytochrome P450 3A5
CYP3A7—cytochrome P450 3A7
DCFH—2′,7′-dichlorodihydrofluorescin
DCFH-DA—2′,7′-dichlorodihydrofluorescin diacetate
DCF—2′,7′-dicholorofluorescein
EDTA—ethylene diamine tetraacetic acid
FDA—United States Food and Drug Administration
HPLC—high performance liquid chromatography
IC₅₀—an inhibitor concentration necessary to inhibit enzyme activity by 50%
LC-UV—liquid chromatography-ultraviolet light [assay]
NADP⁺—nicotinamide adenine dinucleotide phosphate
NADPH—nicotinamide adenine dinucleotide phosphate (reduced)
ROS—reactive oxygen species
UV—ultraviolet

### BACKGROUND

The cytochrome P450 (CYP) system is a superfamily of membrane-bound, heme-containing mixed function oxygenases that are the principal enzyme system for the metabolism of drugs, environmental chemicals, and endogenous compounds (Guengerich, FASEB J 6:667-668 (1992); Eastabrook, FASEB J 10:202-204 (1996); Rendic and Di Carlo, Drug Metab. Rev. 29:413-580 (1997)). These enzymes are expressed in many tissues, however, in mammals are found at the highest levels in liver. Among the 11 drug-metabolizing CYP isoforms expressed in the liver, five of these isoforms are responsible for the metabolism of most pharmaceuticals (CYP1A2, CYP2C9, CYP2C19, CYP2D6 and CYP3A4). Thus, CYPs catalyze the metabolic transformation of drug molecules, which represents a key process by which drugs are cleared from the body.

It is quite common for two or more drugs to be co-administered to a patient, increasing the chances for a drug-drug interaction to occur. Many drug-drug interactions are metabolism-based, and result from two or more drugs competing for the same enzyme with the majority of these interactions involving CYP. Thus, much higher plasma concentrations of a drug are attained if a second drug competes for the enzyme responsible for metabolic clearance of the first drug. For a drug with a narrow therapeutic index, this would lead to an adverse reaction. The significance surrounding this issue is more apparent when considering that there are an estimated two million serious adverse drug reactions that occur per year in the United States, of which 100,000 are fatal (Brennan, Chem Eng News 5:63-73 (2000); see also the United States Food and Drug Administration (FDA) website under “Preventable Adverse Drug Reactions: A Focus on Drug Interactions”). These problems have prompted the need to assess drug safety early during drug discovery/development, and to identify and eliminate compounds that may exhibit a potential for undesirable drug interactions. Assessing the safety of new drug candidates during drug discovery can save considerable amount of time and money, and prevent the exposure of patients to unnecessary risk, especially if a drug must later be removed from the market due to safety issues.

Several types of assays are available for assessing CYP inhibition and use a variety of strategies to evaluate the inhibitory potential of new drug candidates including; (1) liquid chromatography/mass spectrometry, (2) fluorescence-based approaches, and (3) radioisotope-based approaches. Crespi et al. (Anal. Biochem. 248:188-190 (1997)) describe a CYP inhibitor assay that utilizes microtiter plate-based fluorometric methods to assess inhibition of several major xenobiotic-metabolising CYP isoenzymes, such as CYP3A4 and CYP1A2. Similar assays are also described in Kennedy and Jones, Anal. Biochem. 222:217-223 (1994) and in Donato et al., Anal. Biochem. 213:29-33 (1993). Using these assays, the ability of potential drug candidates to interact with CYPs can be ranked based on the relative inhibitory effect on metabolism of the model substrates to produce a fluorogenic product. These assays are relatively fast as compared to other known methods, such as high performance liquid chromatography (HPLC). However, these assays suffer from significant limitations in that they require the use of particular probe substrate, and thus cannot be easily adapted to assess the ability of a given candidate compound to inhibit CYP metabolic activity towards multiple substrates. These assays are further limited by the fact that certain CYPs, including CYP3A4, exhibit dramatically different inhibition profiles (for example, as measured by IC₅₀ values) when the same inhibitor is used with different substrates. Thus, current CYP inhibition assays necessitate the use of particular probe substrates, and do not allow the flexibility of selecting different substrates.

Therefore, an improved assay is needed, such as an assay that allows for the screening of many compounds for an ability to inhibit metabolism of substrates by CYP in a single effort.

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to some embodiments of the presently disclosed subject matter, whether listed in this Summary or not. To avoid excessive repetition, this Summary does not list or suggest all possible combinations of such features.

The presently disclosed subject matter provides a method of screening a candidate compound for an ability to inhibit a cytochrome P450. In some embodiments, the method comprises (a) providing a cytochrome P450 substrate and a candidate compound suspected of having an ability to inhibit a cytochrome P450; (b) mixing the candidate compound, the cytochrome P450 substrate, an indicator compound precursor, the cytochrome P450, and NADPH or an NADPH regenerating system, wherein a primary metabolic activity of the cytochrome P450 produces a chemical species in a side reaction; (c) producing the chemical species in the side reaction, whereby a fraction of the chemical species reacts with the indicator compound precursor to produce an indicator compound; and (d) comparing the amount of indicator compound produced in the presence of the candidate compound to an amount of indicator produced in the absence of the candidate compound, the comparing indicating the ability of the candidate compound to inhibit the cytochrome P450. In some embodiments, the chemical species produced in the side reaction by the primary metabolic activity of the enzyme comprises a reactive oxygen species. In some embodiments, the indicator compound precursor is selected from the group consisting of a fluorogenic compound, a colorimetric compound, a chemiluminescent compound and combinations thereof. In some embodiments, the indicator compound precursor is a fluorogenic compound. In some embodiments, steps (a) through (c) are carried out in at least one well of a multi-well plate.

In some embodiments, the presently disclosed method further comprises screening a plurality of candidate compounds simultaneously for an ability to inhibit a cytochrome P450. In some embodiments, steps (a) through (c) are carried out in multiple wells of a multi-well plate.

In some embodiments of the instant method, the cytochrome P450 is selected from the group consisting of a CYP1A, a CYP2B, a CYP2C, a CYP2D, a CYP2E, a CYP3A, and combinations thereof. In some embodiments, the substrate is selected from the group consisting of a CYP1A substrate, a CYP2B substrate, a CYP2C substrate, a CYP2D substrate, a CYP2E substrate, a CYP3A substrate, and combinations thereof. In some embodiments, the CYP3A4 substrate is selected from the group consisting of testosterone, midazolam, quinidine, and verapamil; the CYP1A2 substrate is phenacetin; the CYP2C9 substrate is selected from the group consisting of diclofenac and tolbutamide; the CYP2D6 substrate is selected from the group consisting of bufuralol, imipramine, and dextromethorphan; the CYP2C19 substrate is selected from the group consisting of omeprazole and S-mephenytoin; and/or the CYP2E1 substrate is chlorzoxazone.

In some embodiments, the cytochrome P450 comprises a human cytochrome P450. In some embodiments, the human cytochrome P450 is selected from the group consisting of CYP1A2, CYP2B6, CYP2C8, CYP2C9, CYP2C19, CYP2D6, CYP2E1, CYP3A4, CYP3A5, CYP3A7, and combinations thereof. In some embodiments, the human cytochrome P450 is CYP3A4.

In some embodiments, the instant method further comprises quantifying an amount of indicator compound produced in the presence of the candidate compound. In some embodiments, the instant method further comprises quantifying an amount of indicator compound formed over a period of time to thereby determine an inhibitory potency of the candidate compound for the cytochrome P450.

The presently disclosed subject matter also provides a method of determining an inhibitory potency of a candidate compound for a cytochrome P450. In some embodiments, the method comprises (a) providing a cytochrome P450 substrate and a candidate compound suspected of having an ability to inhibit a cytochrome P450; (b) mixing the candidate compound, the cytochrome P450 substrate, an indicator compound precursor, the cytochrome P450, and NADPH or an NADPH regenerating system, wherein a primary metabolic activity of the cytochrome P450 produces a chemical species in a side reaction; (c) producing the chemical species in the side reaction, whereby a fraction of the chemical species reacts with the indicator compound precursor to produce an indicator compound; (d) quantifying a rate at which the indicator compound is produced in the presence of the candidate compound; and (e) determining a concentration of the candidate compound that reduces the primary metabolic activity of the cytochrome P450 by a pre-set amount, whereby the inhibitory potency of the compound for a cytochrome P450 is determined.

In some embodiments of the instant method, the cytochrome P450 is selected from the group consisting of a CYP1A, a CYP2B, a CYP2C, a CYP2D, a CYP2E, a CYP3A, and combinations thereof. In some embodiments, the substrate is selected from the group consisting of a CYP1A substrate, a CYP2B substrate, a CYP2C substrate, a CYP2D substrate, a CYP2E substrate, a CYP3A substrate, and combinations thereof. In some embodiments, the CYP3A4 substrate is selected from the group consisting of testosterone, midazolam, quinidine, and verapamil. In some embodiments, the CYP1A2 substrate is phenacetin. In some embodiments, the CYP2C9 substrate is selected from the group consisting of diclofenac and tolbutamide. In some embodiments, the CYP2D6 substrate is selected from the group consisting of bufuralol, imipramine, and dextromethorphan. In some embodiments, the CYP2C19 substrate is selected from the group consisting of omeprazole and S-mephenytoin. In some embodiments, the CYP2E1 substrate is chlorzoxazone.

In some embodiments of the instant method, the cytochrome P450 comprises a human cytochrome P450. In some embodiments, the human cytochrome P450 is selected from the group consisting of CYP1A2, CYP2B6, CYP2C8, CYP2C9, CYP2C19, CYP2D6, CYP2E1, CYP3A4, CYP3A5, CYP3A7, and combinations thereof. In some embodiments, the human cytochrome P450 is CYP3A4.

In some embodiments, the chemical species produced in the side reaction by the primary metabolic activity of the enzyme comprises a reactive oxygen species. In some embodiments, the indicator compound precursor is selected from the group consisting of a fluorogenic compound, a colorimetric compound, a chemiluminescent compound, and combinations thereof. In some embodiments, the indicator compound precursor is a fluorogenic compound.

In some embodiments of the instant method, steps (a) through (c) are carried out in at least one well of a multi-well plate. In some embodiments, steps (a) through (c) are carried out in multiple wells of a multi-well plate.

In some embodiments, the pre-set amount is selected from the group consisting of 25%, 33%, 50%, 67%, 75%, and 90%.

In some embodiments, the instant method further comprises determining inhibitory potencies of a plurality of candidate compounds simultaneously. In some embodiments, the instant method further comprises quantifying a rate at which the indicator compound is produced in the presence of the candidate compound.

Accordingly, it is an object of the presently disclosed subject matter to provide a method for screening a candidate compound for an ability to inhibit a cytochrome P450. This object is achieved in whole or in part by the presently disclosed subject matter.

An object of the presently disclosed subject matter having been stated herein above, other objects will become evident as the description proceeds as best described herein below.

### BRIEF DESCRIPTION OF THE DRAWINGS

FIG. 1 schematically depicts the secondary or side reaction associated with cytochrome P450 metabolism that produces reactive oxygen species (for example, OH⁻, O₂⁻ and H₂O₂). These reactive oxygen species are then available for reacting with indicator precursor compounds to produce indicator compounds, which can be detected using various standard techniques.

FIG. 2 schematically depicts the use of a representative indicator precursor compound, dichlorodihydrofluorescin-diacetate (DCFH-DA), to detect ROS production during CYP catalysis. DCHFH-DA can traverse cell membranes and be subsequently deacetylated by cellular esterases to form dichorodihydrofluorescein (DCFH). DCFH is highly reactive towards ROS forming the fluorescent product, dichlorofluorescein (DCF).

FIG. 3 depicts (i) fluorescence production during CYP3A4-mediated metabolism of testosterone, dextromethorphan, and quinidine, and (ii) the effect of ketoconazole (a CYP3A4 inhibitor) on the inhibition of fluorescence production. Solid bars represent fluorescence production in the presence of ketoconazole, and hatched bars represent fluorescence production in the absence of ketoconazole.

FIGS. 4A and 4B, 5A and 5B, 6A and 6B, and 7A and 7B depict the inhibition of CYP3A4-mediated testosterone metabolism by ketoconazole, nifedipine, progesterone, and quinidine, respectively. For each set of Figures, the Figure A depicts the calculation of IC₅₀ values using the conventional LC-UV method for quantifying product (6β-hydroxytestosterone) formation, and the Figure B depicts the calculation of IC₅₀ values using the ROS-based fluorescence assay disclosed herein. In each Figure, the X-axis represents inhibitor concentration plotted on a logarithmic scale, and the y-axis depicts the percent fluorescence detected, with the amount of fluorescence detected in the absence of inhibitor set at 100%.

### DETAILED DESCRIPTION

I. General Considerations

Among human CYPs, CYP3A4 affects metabolic fate of more drugs than any other CYP. As a consequence, a potent inhibitor of this enzyme could have a significant impact on the disposition of many drugs that are likely to be co-administered. Hence, many pharmaceutical companies have implemented screens to assess the inhibitory potency of drug candidates toward CYP3A4. These assays include liquid chromatography-ultraviolet light (LC-UV), fluorescence-based, and radioisotope-based approaches. Typically, inhibition of a CYP by candidate compounds is assessed against a specific probe substrate based upon the assumption that the inhibitory potency of a test compound is independent of the probe substrate being used to assess CYP activity. This assumption is not accurate, however, and CYP3A4 in particular is an exception in that it displays multiple IC₅₀ values for the same inhibitor when using different substrates, which might be due to the ability of this isoform to simultaneously bind multiple substrates within its active site. Existing fluorescence-based and radiometric assays to assess CYP inhibition require the use of specific, non-“drug-like”, probe substrates, and thus do not allow the flexibility of selecting different and/or multiple substrates.

II. Definitions

Unless otherwise defined, all technical and scientific terms used herein have the same meaning as commonly understood by one of ordinary skill in the art to which the presently disclosed subject matter pertains. For clarity of the present specification, certain definitions are presented herein below.

While the following terms are believed to be well understood by one of skill in the art, the following definitions are set forth to facilitate explanation of the presently disclosed subject matter.

Following long-standing patent law convention, the terms “a” and “an” mean “one or more” when used in this application, including the claims. Thus, for example, the term “a substrate” is understood herein to refer to one or more substrates, and “a CYP2C” is understood herein to refer to one or more members of the CYP2C subfamily.

In some embodiments of the presently disclosed subject matter, a method is provided for the screening of a candidate compound for an ability to inhibit a cytochrome P450. The method comprises reacting a candidate compound suspected of having an ability to inhibit a cytochrome P450 (CYP), the CYP substrate, and an indicator compound precursor, with the CYP under conditions that support CYP biological activity (in some embodiments in the presence of NADPH or an NADPH regenerating system), wherein a primary metabolic activity of the CYP produces a chemical species in a side reaction; producing the chemical species in the side reaction, whereby a fraction of the chemical species reacts with the indicator compound precursor to produce an indicator compound; and comparing the amount of indicator compound produced in the presence of the candidate compound to an amount of indicator produced under identical conditions in the absence of the candidate compound. By comparing the amount of indicator substance produced in the presence of the candidate compound to the amount produced in its absence, the ability of the candidate compound to inhibit the CYP is determined. In some embodiments, the method comprises quantifying an amount of indicator compound produced in the presence of the candidate compound.

The term “candidate compound” is meant to refer to any compound wherein the characterization of the compound's ability to inhibit a CYP is of interest. As such, the terms “candidate compound” and “cytochrome P450 inhibitor” are used interchangeably herein. Exemplary candidate compounds include xenobiotics such as drugs, and other therapeutic agents including, but not limited to small molecules. Representative candidate compounds include ketoconazole, nelfinavir, cyclosporine, saquinavir, nifedipine, diltiazem, omeprazole, progesterone, erythromycin, verapamil, and prednisolone.

The terms “cytochrome P450” and “CYP” are meant to refer to a large family (often called a “superfamily”) of hemoprotein enzymes capable of metabolizing xenobiotics such as drugs, carcinogens, and environmental pollutants, as well as endobiotics such as steroids, fatty acids, and prostaglandins. As used herein, these terms are meant to encompass all members of the CYP superfamily, regardless of the species of origin. Thus, these terms are meant to refer to CYP superfamily members of microbial, invertebrate, and vertebrate origin. In some embodiments, these terms refer to CYP's of warm-blooded vertebrate species, including birds and mammals. Representative sources of CYPs thus include bovine, porcine, ovine, canine, feline, equine, murine and avian sources. In some embodiments, these terms refer to CYPs of human origin.

All isoenzymes, or isoforms, within the CYP superfamily are contemplated to fall within the terms “cytochrome P450” and “CYP” as used herein. Particularly contemplated CYP isoforms include, but are not limited to, members of the CYP1A, CYP2B, CYP2C, CYP2D, CYP2E, and CYP3A families, as these isoforms have been identified as those most commonly responsible for the metabolism of drugs in humans. Additional CYP superfamily members are described in U.S. Pat. Nos. 5,786,191 and 5,478,723, the contents of each of which are herein incorporated by reference.

The terms “chemical species” and “chemical byproduct” are meant to refer to a species or byproduct that is formed from a side or secondary reaction associated with the primary metabolic activity of a selected enzyme. The terms “chemical species” and “chemical byproduct” are further characterized in that the species or byproduct produced from the secondary or side reaction associated with the metabolic activity of the enzyme is capable of reacting with an indicator compound precursor in accordance with the methods of the presently disclosed subject matter.

Exemplary chemical species comprise the “reactive oxygen species” or “ROS” produced from side reactions of several enzymes, including CYP-superfamily enzymes. Exemplary ROS comprise superoxide anion (O₂⁻), hydrogen peroxide (H₂O₂), and hydroxyl radical (OH⁻).

The term “indicator compound precursor” is meant to refer to a chemical compound that reacts with the chemical species produced from the side reaction associated with metabolic activity of the enzyme. The term “indicator compound” is meant to refer to the compound produced by the reaction of the indicator compound precursor and the chemical species. As noted above, the presence of the indicator compound indicates the susceptibility of the candidate compound to metabolism by the selected enzyme. The preferred indicator compound is readily detectable using a standard detection technique, such as fluorescence or chemiluminescence spectrophotometry, colorimetry, and the like.

Exemplary indicator compound precursors thus include, but are not limited to, compounds that are converted to fluorogenic/fluorescent compounds, chemiluminescent compounds, colorimetric indicator compounds, and combinations thereof. A particularly contemplated indicator compound precursor/indicator compound system comprises the fluorogenic probe 2′,7′-dichlorodihydrofluorescin-diacetate (DCFH-DA) and its fluorescent counterpart, 2′,7′-dichlorofluorescein (DCF). Other indicator compound precursor/indicator compound systems can be used, however, including, but not limited to dihydrorhodamine 123/rhodamine 123 and dihydroethidium/ethidum,

In some embodiments of the presently disclosed subject matter, a CYP is reacted with a CYP substrate. As used herein, the phrase “CYP substrate” refers to any substrate that can bind to and be acted upon as a result of a primary metabolic activity of a CYP that produces a chemical species in a side reaction. Exemplary CYP substrates include testosterone, midazolam, quinidine, and verapamil (CYP3A4); phenacetin (CYP1A2); diclofenac and tolbutamide (CYP2C9); bufuralol, imipramine, and dextromethorphan (CYP2D6); omeprazole and S-mephenytoin (CYP2C19); and chlorzoxazone (CYP2E1). Other medically relevant cytochrome P450 substrates, as well as cytochrome P450 inhibitors and inducers, which can be employed in the methods of the presently disclosed subject matter include, but are not limited to those listed on a World Wide Web page maintained by Dr. David Flockhart of the Indiana University School of Medicine and accessible from the Indiana University-Purdue University Indianapolis webpage (search “drug interactions”). It is also provided that a particular molecule could act as an inhibitor under certain circumstance and as a substrate under certain circumstances. For example and particularly in the case of certain drug-drug interactions, one drug can be considered a substrate of a CYP because it binds to and is metabolized by the CYP, and the same drug can be considered an inhibitor when present in conjunction with a second drug because its interaction with the CYP decreases the ability of the same CYP to metabolize the second drug or the rate at which the second drug is metabolized. This can occur by any mechanism, for example when the first and second drugs bind to the same active site with the first drug having a higher binding affinity, or when the first and second drugs bind to different active sites but the binding of the first drug to its active site renders inaccessible the active site to which the second drug would have bound.

As used herein, NADPH refers to the reduced form of nicotinamide adenine dinucleotide phosphate, a cofactor for CYP activity. This compound can either be added directly to the reaction or produced using an NADPH regenerating system. The regenerating system consists of NADP⁺, and an enzyme system that is capable of reducing this compound to NADPH. Exemplary NADPH regenerating systems comprise NADP⁺, glucose-6-phosphate, and glucose-6-phosphate dehydrogenase; however, the regenerating enzyme/substrate system is not limited to these in particular. Thus, in some embodiments of the presently disclosed subject matter, an NADPH regenerating system comprises a component of the reaction conditions that support CYP biological activity, and in some embodiments an NADPH regenerating system comprises a plurality of components of the reaction conditions that support CYP biological activity.

As used herein, the term “compare”, and grammatical variants thereof, can refer to evaluating an amount of an indicator compound produced. As such, the term refers to a quantitative measurement (for example, a number of fluorescence units detected) or qualitative measurement (for example, visually or otherwise relating one experiment to one or more other as producing more or less of an indicator compound). Additionally, comparing refers to measurements relative to a standard. For example, comparing can refer to a measurement of an output (for example, fluorescence) in the presence of an inhibitor relative to a measurement of the same output in the absence of an inhibitor, wherein the latter can be set to 100% and the former determined as a percentage of the latter. In some embodiments, comparing can also refer to a simple relative measurement such as “darker” or “lighter”, or “more” or “less”.

As used herein, the term “quantify”, and grammatical variants thereof, refers to determining an amount of an indicator compound produced. As such, the term can refer to a quantitative measurement (for example, a number of fluorescence units detected). Additionally, quantifying can refers to determining an amount relative to a standard.

As used herein, the term “inhibit”, when used in relation to a candidate compound in a reaction with a CYP, refers to an ability of the candidate compound to cause a decrease in the activity of a CYP. It is understood, however, that under certain conditions (for example, very low concentrations of the inhibitor) an inhibitor might cause an increase in the activity of a CYP. Under these circumstances, a candidate compound is considered an inhibitor if, after reaching some minimum concentration, the candidate compound is able to cause a decrease in the amount of an indicator compound precursor that is converted to an indicator compound.

In some embodiments of the presently disclosed subject matter, a method is provided for determining an inhibitory potency of a candidate compound for a CYP. In some embodiments, the method comprises providing a CYP substrate and a candidate compound suspected of having an ability to inhibit a CYP; mixing the candidate compound, the CYP substrate, an indicator compound precursor, and the CYP under conditions that support CYP biological activity (in some embodiments in the presence of NADPH or an NADPH regenerating system), wherein a primary metabolic activity of the CYP produces a chemical species in a side reaction; producing the chemical species in the side reaction, whereby a fraction of the chemical species reacts with the indicator compound precursor to produce an indicator compound; and determining a concentration of the candidate compound that reduces the primary metabolic activity of the CYP by a pre-set amount, whereby the inhibitory potency of the compound for a CYP is determined. In some embodiments, the method further comprises quantifying a rate at which the indicator compound is produced in the presence of the candidate compound.

As used herein, the term “inhibitory potency” refers to an ability of a candidate compound to inhibit a CYP, for example, by a pre-set amount. As used herein, the phrase “pre-set amount” refers to a pre-determined amount relative to the activity of the CYP in the absence of the candidate compound. A pre-set amount can be, for example, a percentage of the activity of the CYP. Representative pre-set amounts include, but are not limited to 25%, 33%, 50%, 67%, 75%, and 90% of the activity of the CYP under a given set of conditions. In some embodiments, inhibitory potency refers to the concentration of the candidate compound required to reduce the activity of the CYP by 50% under a given set of conditions (i.e. the IC₅₀ value).

It is contemplated that the method of the presently disclosed subject matter can be performed within standard multi-well assay plates as are well known in the art, such as 96-well micro-titer plates. Thus, a plurality of candidate compounds can be simultaneously screened for an ability to inhibit a CYP within multiple wells of a multi-well plate. Additionally, the use of multi-well plates or other multiple reservoir devices can allow for several or many concentrations of an individual candidate compound to be assayed at the same time with either the same or different substrates and/or CYP superfamily members.

As would be apparent to one of skill in the art from the disclosure of the presently disclosed subject matter, it is also contemplated that the methods of the presently disclosed subject matter can be performed in a cell-free reaction and/or in a cell-based, in vitro reaction. Exemplary cell-based, in vitro platforms suitable for modification in accordance with the method of the presently disclosed subject matter are described in Parkinson, Toxicol. Pathol. 24:45-57 (1996). As would also be apparent to one of skill in the art from the disclosure of the presently disclosed subject matter, the methods of the presently disclosed subject matter are contemplated to be useful for other combinations of indicator compound precursor-indicator compound. For example, Ferric-EDTA is contemplated to be an indicator compound precursor that can give rise to a chemiluminescent product in the presence of a reactive oxygen species (Puntarulo and Cederbaum, Arch. Biochem. Biophys. 258: 510-518 (1987)).

### EXAMPLE

The following Example has been included to illustrate representative modes of the presently disclosed subject matter. In light of the present disclosure and the general level of skill in the art, those of skill will appreciate that the following Example is intended to be exemplary only and that numerous changes, modifications, and alterations can be employed without departing from the spirit and scope of the presently disclosed subject matter.

### Materials Used in the Example

1′-hydroxymidazolam and microsomes (SUPERSOMES™) prepared from baculovirus-infected insect cells containing recombinant heterologously expressed human CYP plus NADPH CYP reductase were purchased from GenTest Corp. (Woburn, Mass., United States of America). 6β-hydroxytestosterone and 11α-hydroxyprogesterone were obtained from Steraloids Inc. (Wilton, N.H., United States of America). Testosterone, midazolam, ketoconazole, itraconazole, miconazole, furafylline, nifedipine, verapamil, progesterone, diltiazem, erythromycin, prednisolone, cyclosporine A, omeprazole, quinidine, sulfaphenazole, β-nicotinamide adenine dinucleotide phosphate reduced tetrasodium salt β-NADPH), and 2′,7′-dichlorodihydrofluorescein diacetate were purchased from Sigma Chemical Co. (St. Louis, Mo., United States of America). 2′,7′-dichlorofluorescein (DCF) was obtained from Aldrich Chemical Co. (Milwaukee, Wis., United States of America).

### Inhibition of CYP3A4-mediated Oxidation of Testosterone as Measured by Dichlorodihydrofluorescein (DCFH-DA) Oxidation

Stock solutions of DCFH-DA (2.5 mM) and NADPH (20 mM) were prepared prior to each experiment in methanol and buffer, respectively. Substrates and inhibitors were prepared in 100% methanol and stored at −20° C. Unless otherwise specified, reaction mixtures included 2.5 μM DCFH-DA, 50 pmol/ml CYP microsomes, and 50 mM Tris or 100 mM potassium phosphate buffer (pH 7.4 supplemented with 3.3 mM MgCl₂) to a final volume of 200 μl. Unless otherwise indicated, testosterone were added to the reactions at a final concentration of 40 μM. An equal volume of methanol was added to samples containing no substrate and served as controls. Reactions were performed at 37° C. in 96-well black bottom plates. Inhibitors were added at varying concentrations from stock solutions prepared in either water or methanol. Final methanol concentrations were kept constant among samples (1%, v/v) to eliminate variability in the rate of fluorescence production due to the effect of methanol on CYP activity. Reactions were preincubated for five minutes at 37° C. in the dark without shaking prior to the addition of pre-warmed NADPH (1 mM final concentration). Fluorescence was measured over a one hour period at five minute intervals using a Molecular Devices Microplate Fluorescence Reader (Molecular Devices Corp., Sunnyvale, Calif., United States of America) with the following parameters; excitation wavelength=500 nm, emission wavelength=529 nm, optical position=top, temperature=37° C., plate format=black bottom 96-well. The concentration of DCF in the reactions was determined using a standard curve constructed with authentic DCF.

Inhibition of CYP3A4-mediated oxidation of testosterone was measured by 6β-hydroxytestosterone formation. The kinetics of 6β-hydroxytestosterone formation by CYP3A4 was determined and compared to rate of fluorescence production. Reactions consisted of varying concentrations of testosterone, 50 pmol CYP3A4 per ml, and buffer to a final volume of one milliliter. Reactions were performed at 37° C. and were started upon addition of 1 mM NADPH. Aliquots (200 μl) were removed at 0, 10, and 30 minutes after the addition of NADPH and stopped with the addition of 100 μl of acetonitrile containing 4.5 μM of 11α-hydroxyprogesterone (HPLC internal standard). Samples were placed immediately on ice and the precipitated protein was removed by centrifugation for four minutes at 13,000 rpm. The supernatant was transferred to HPLC vials for analysis.

HPLC analysis was performed using an Agilent 1100 Series HPLC system (Agilent Technologies, Palo Alto, Calif., United States of America) equipped with an online UV-detector. Testosterone and its metabolite, 6β-hydroxytestosterone, were separated on a Keystone Aquasil C18 column (150×4.6 mm, 5 μm particle size; Keystone Scientific Inc., Bellefonte, Pa., United States of America) maintained at 28° C. A fifteen minute gradient was applied to the column at a flow rate of one milliliter per minute starting with a mobile phase of water/acetonitrile/methanol (63:2:35 v/v) and ending with water/acetonitrile/methanol (18:2:80 v/v), followed by three minutes of isocratic conditions using the latter buffer. Analytes were detected and quantified at 242 nm. The retention times for testosterone, 6β-hydroxytestosterone, and 11α-hydroxyprogesterone were about 10.8, 13.1, and 15.4 minutes, respectively, and verified using authentic standards. All experiments were performed in triplicate.

### Data Analysis

IC₅₀ values (inhibitor concentration resulting in 50% reduction in CYP activity) were calculated using XLfit 4 (ID Business Solutions, Inc., Emeryville, Calif., United States of America). Data were fitted to a sigmoidal dose-response model.

### Discussion of the Example

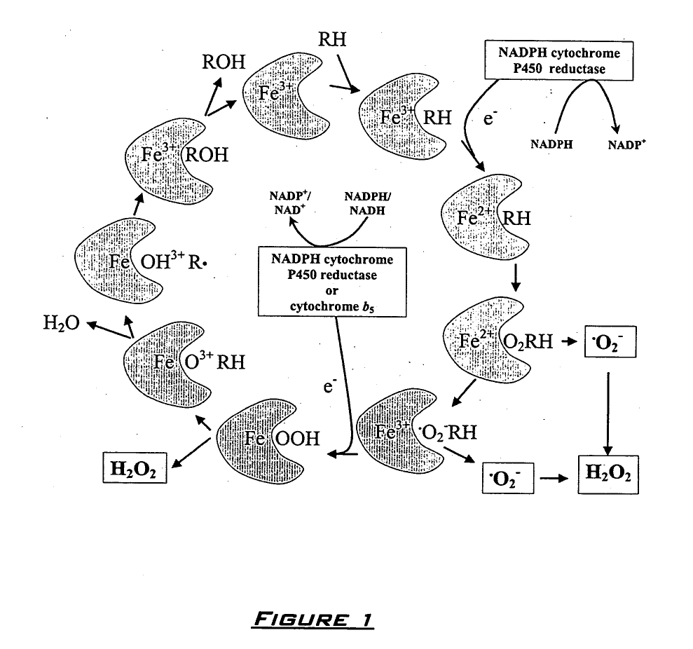

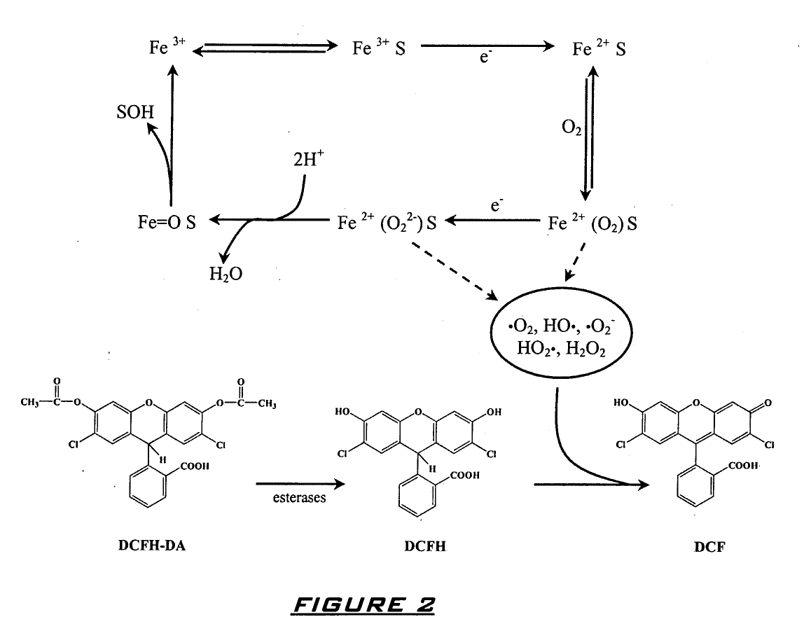

Interaction of dichlorodihydrofluorescin with reactive oxygen species produced during CYP metabolism. Reactive oxygen species (ROS) have long been identified as a by-product of the cytochrome P450 (CYP) catalytic cycle and result from the dissociation of activated oxygen prior to its incorporation into substrate or reduction to water (see FIG. 1). A technology using dichlorodihydrofluorescin-diacetate (DCFH-DA) to detect ROS production during CYP catalysis is described in U.S. Pat. No. 6,312,917. DCFH-DA is a lipophilic molecule that can easily traverse cellular membranes and be subsequently deacetylated by cellular esterases to form dichorodihydrofluorescein (DCFH). DCFH is highly reactive towards ROS and thus forms the highly fluorescent product, dichlorofluorescein (DCF; see FIG. 2).

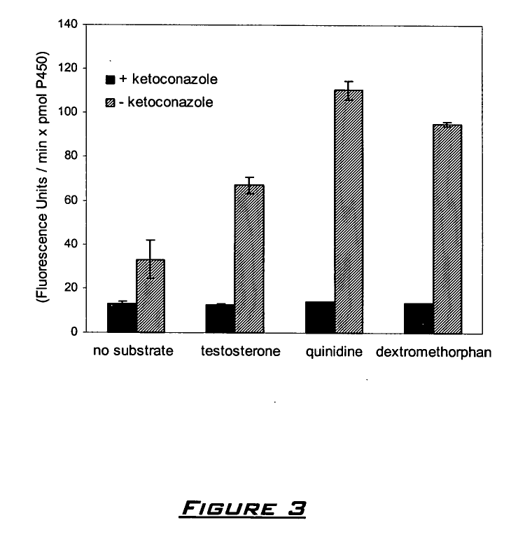

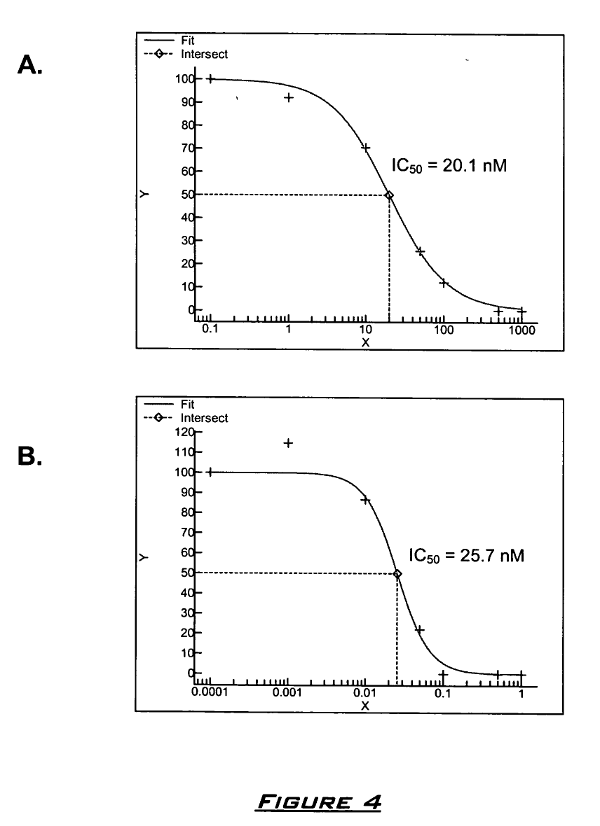

DCF formation during CYP3A4-mediated testosterone hydroxylation. DCFH can serve as a ROS-sensitive fluorogenic probe for measuring the metabolic activity of CYP catalyzed reactions (FIG. 2). However, the presently disclosed subject matter expands on the use of this technology to identify CYP inhibitors by quantifying the reduction of fluorescence (i.e., DCF formation) in the presence of (1) a CYP substrate (for example, testosterone or other CYP substrate); and (2) a CYP inhibitor that affects the rate of fluorescence production. Thus, a reduction in CYP-derived fluorescence production during substrate (for example, testosterone or other CYP substrate) metabolism is indicative of a putative inhibitor inhibiting the oxidative metabolism of the substrate. The addition of ketoconazole, a potent CYP3A4 inhibitor, to reactions containing recombinant CYP3A4 microsomes (CYP3A4 and NADPH-CYP reductase), NADPH, DCFH-DA, and testosterone, resulted in a reduction in fluorescence production as depicted in FIGS. 3, 4A, and 4B. The addition of ketoconazole to CYP3A4 reactions containing other CYP3A4 substrates such as dextromethorphan and quinidine also resulted in a significant reduction in fluorescence signal (see FIG. 3).

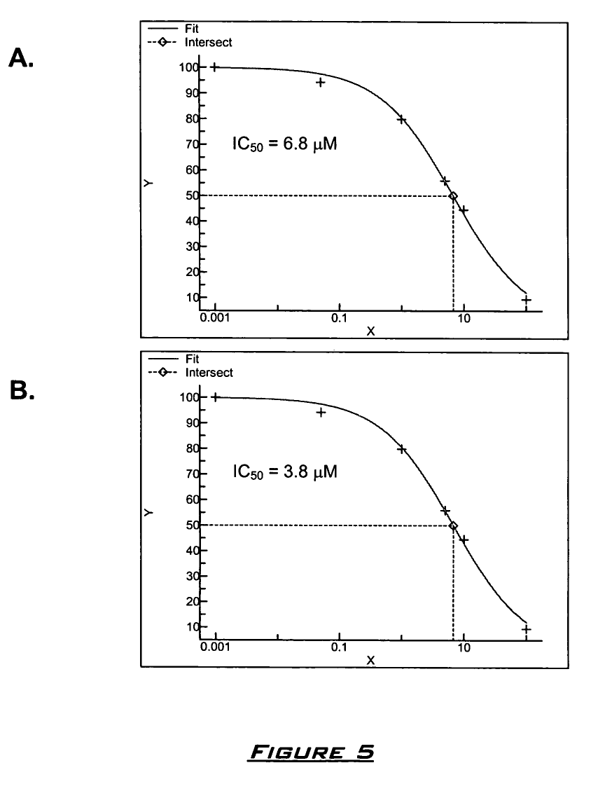

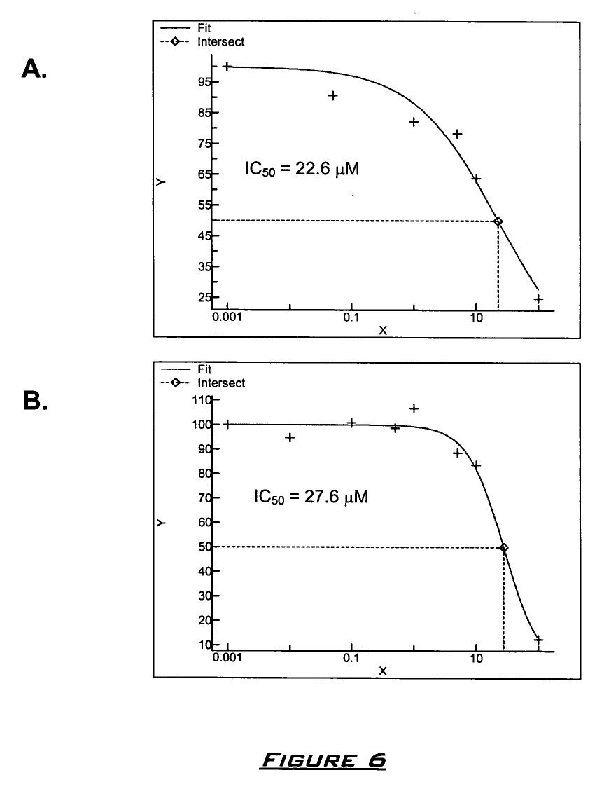

Determining IC₅₀ values for putative CYP3A4 inhibitors using the ROS-based fluorescent assay. IC₅₀ (inhibitor concentration resulting in 50% reduction in enzyme activity) values were calculated for 13 compounds. Eleven of these compounds are known CYP3A4 inhibitors and two compounds served as negative controls that display no known inhibitory activity towards CYP3A4. IC₅₀ values were determined using the ROS-based fluorescence assay disclosed herein and these results were compared to IC₅₀ values calculated using the conventional approach of quantifying 6β-hydroxytestosterone (LC-UV quantification of 6β-hydroxytestosterone formation). Ketoconazole, one of the most potent CYP3A4 inhibitors, resulted in a concentration dependent decrease in fluorescence production in the presence of DCFH and testosterone (see FIGS. 4A and 4B). IC₅₀ values for ketoconazole inhibition were 25.7 nM and 20.1 nM using the ROS-based fluorescence assay and the conventional assay, respectively (see FIGS. 4A and 4B and Table 1). IC₅₀ values are presented in Table 1 for the 13 compounds tested using both assays and are listed in order with respect to their inhibitory potency using the fluorescent assay. Besides ketoconazole, six compounds were classified as moderately potent inhibitors of CYP3A4 activity with an IC₅₀ value below 20 μM as determined using the fluorescent assay. Cyclosporin was the only inhibitor among this group of compounds that showed poor correlation to the IC₅₀ value generated using the conventional assay. The concentration dependent decrease in fluorescence vs. 6β-hydroxytestosterone formation for nifedipine is shown in FIGS. 5A and 5B (IC₅₀ values of 6.8 μM and 3.8 μM for the ROS-based fluorescence assay and the conventional assay, respectively), and for progesterone is shown in FIGS. 6A and 6B (IC₅₀ values of 22.6 μM and 27.6 μM for the ROS-based fluorescence assay and the conventional assay, respectively).

TABLE 1
IC₅₀ Values Obtained using the ROS-based Fluorescence
Assay vs. a Conventional Assay

| Compound | ROS-based IC₅₀ (μM) | LC-based IC₅₀ (μM) |
|---|---|---|
| Ketoconazole | 2.6 x 10⁻² | 2.0 x 10⁻² |
| Nelfinavir | 1.2 | 0.4 |
| Cyclosporin | 1.3 | 63.9 |
| Saquinavir | 3.6 | 1.0 |
| Nifedipine | 3.8 | 6.8 |
| Diltiazem | 10.2 | 22.3 |
| Omeprazole | 15.3 | 39.5 |
| Progesterone | 27.6 | 22.6 |
| Erythromycin | 27.6 | 1.7 |
| Verapamil | 30.0 | 7.0 |
| Prednisolone | 79.0 | >100 |
| Quinidine | >100 | 98.2 |
| Sulfaphenazole | >100 | >100 |

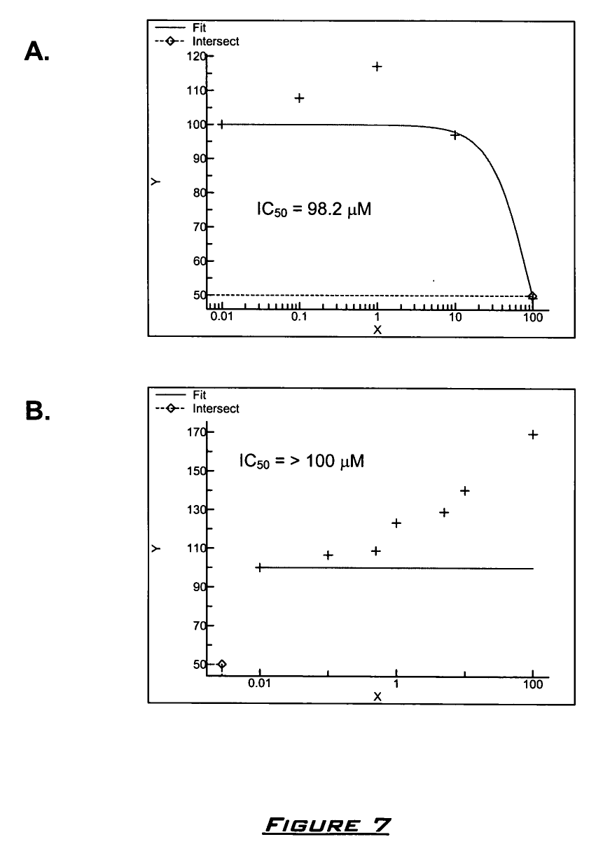

Four out of the thirteen compounds, including progesterone, erythromycin, verapamil, and prednisolone were weak inhibitors of CYP3A4 activity based on the IC₅₀ values calculated using the fluorescence assay. Erythromycin was the only compound among this group of inhibitors that showed a poor correlation between the fluorescent and conventional IC₅₀ values. Both quinidine (see FIGS. 7A and 7B) and sulfaphenazole showed substantially no inhibitory activity using either approach (as determined by having an IC₅₀ value of about 100 μM or greater).

### REFERENCES

The references listed below as well as all references cited in the specification are incorporated herein by reference to the extent that they supplement, explain, provide a background for or teach methodology, techniques and/or compositions employed herein.

Birkett et al., Trends Pharmacol. Sci. 14:151-185(1993).

Brennan, Chem Eng News 5:63-73 (2000).

Crespi et al., Anal. Biochem. 248:188-190 (1997).

Donato et al., Anal. Biochem. 213:29-33 (1993).

Eastabrook, FASEB J 10:202-204 (1996).

Guengerich, FASEB J 6:667-668 (1992).

Guengerich and Shimada, Chem. Res. Toxicol. 4:391-407 (1991).

Kennedy and Jones, Anal. Biochem. 222:217-223 (1994).

Parkinson, Toxicol. Pathol. 24:45-57 (1996).

Puntarulo and Cederbaum, Arch. Biochem. Biophys. 258: 510-518 (1987).

Rendic and Di Carlo, Drug Metab. Rev. 29:413-580 (1997).

Spatnegger and Jaeger, Drug Metab. Rev. 27:397-417 (1995).

U.S. Pat. No. 5,478,723

U.S. Pat. No. 5,786,191

U.S. Pat. No. 6,312,917.

Wrighton et al., Drug Metab. Rev. 25:453-484 (1993).

It will be understood that various details presented herein can be changed without departing from the scope of the presently disclosed subject matter. Furthermore, the foregoing description is for the purpose of illustration only, and not for the purpose of limitation.

What is claimed is:

1. A method of screening a candidate compound for an ability to inhibit a cytochrome P450, the method comprising:
(a) providing a cytochrome P450 substrate and a candidate compound suspected of having an ability to inhibit a cytochrome P450;
(b) mixing the candidate compound, the cytochrome P450 substrate, an indicator compound precursor, the cytochrome P450, and NADPH or an NADPH regenerating system, wherein a primary metabolic activity of the cytochrome P450 produces a chemical species in a side reaction;
(c) producing the chemical species in the side reaction, whereby a fraction of the chemical species reacts with the indicator compound precursor to produce an indicator compound; and
(d) comparing the amount of indicator compound produced in the presence of the candidate compound to an amount of indicator produced in the absence of the candidate compound, the comparing indicating the ability of the candidate compound to inhibit the cytochrome P450.

2. The method of claim 1, wherein the chemical species produced in the side reaction by the primary metabolic activity of the enzyme comprises a reactive oxygen species.

3. The method of claim 1, wherein the indicator compound precursor is selected from the group consisting of a fluorogenic compound, a calorimetric compound, a chemiluminescent compound and combinations thereof.

4. The method of claim 3, wherein the indicator compound precursor is a fluorogenic compound.

5. The method of claim 1, wherein steps (a) through (c) are carried out in at least one well of a multi-well plate.

6. The method of claim 1, further comprising screening a plurality of candidate compounds simultaneously for an ability to inhibit a cytochrome P450.

7. The method of claim 6, wherein steps (a) through (c) are carried out in multiple wells of a multi-well plate.

8. The method of claim 1, wherein the cytochrome P450 is selected from the group consisting of a CYP1A, a CYP2B, a CYP2C, a CYP2D, a CYP2E, a CYP3A, and combinations thereof.

9. The method of claim 1, wherein the substrate is selected from the group consisting of a CYP1A substrate, a CYP2B substrate, a CYP2C substrate, a CYP2D substrate, a CYP2E substrate, a CYP3A substrate, and combinations thereof.

10. The method of claim 9, wherein the CYP3A4 substrate is selected from the group consisting of testosterone, midazolam, quinidine, and verapamil.

11. The method of claim 9, wherein the CYP1A2 substrate is phenacetin.

12. The method of claim 9, wherein the CYP2C9 substrate is selected from the group consisting of diclofenac and tolbutamide.

13. The method of claim 9, wherein the CYP2D6 substrate is selected from the group consisting of bufuralol, imipramine, and dextromethorphan.

14. The method of claim 9, wherein the CYP2C19 substrate is selected from the group consisting of omeprazole and S-mephenytoin.

15. The method of claim 9, wherein the CYP2E1 substrate is chlorzoxazone.

16. The method of claim 1, wherein the cytochrome P450 comprises a human cytochrome P450.

17. The method of claim 16, wherein the human cytochrome P450 is selected from the group consisting of CYP1A2, CYP2B6, CYP2C8, CYP2C9, CYP2C19, CYP2D6, CYP2E1, CYP3A4, CYP3A5, CYP3A7, and combinations thereof.

18. The method of claim 17, wherein the human cytochrome P450 is CYP3A4.

19. The method of claim 1, further comprising quantifying an amount of indicator compound produced in the presence of the candidate compound.

20. The method of claim 19, further comprising quantifying an amount of indicator compound formed over a period of time to thereby determine an inhibitory potency of the candidate compound for the cytochrome P450.

21. A method of determining an inhibitory potency of a candidate compound for a cytochrome P450, the method comprising:
(a) providing a cytochrome P450 substrate and a candidate compound suspected of having an ability to inhibit a cytochrome P450;
(b) mixing the candidate compound, the cytochrome P450 substrate, an indicator compound precursor, the cytochrome P450, and NADPH or an NADPH regenerating system, wherein a primary metabolic activity of the cytochrome P450 produces a chemical species in a side reaction;
(c) producing the chemical species in the side reaction, whereby a fraction of the chemical species reacts with the indicator compound precursor to produce an indicator compound;
(d) quantifying a rate at which the indicator compound is produced in the presence of the candidate compound; and
(e) determining a concentration of the candidate compound that reduces the primary metabolic activity of the cytochrome P450 by a pre-set amount, whereby the inhibitory potency of the compound for a cytochrome P450 is determined.

22. The method of claim 21, wherein the cytochrome P450 is selected from the group consisting of a CYP1A, a CYP2B, a CYP2C, a CYP2D, a CYP2E, a CYP3A, and combinations thereof.

23. The method of claim 21, wherein the substrate is selected from the group consisting of a CYP1A substrate, a CYP2B substrate, a CYP2C substrate, a CYP2D substrate, a CYP2E substrate, a CYP3A substrate, and combinations thereof.

24. The method of claim 23, wherein the CYP3A4 substrate is selected from the group consisting of testosterone, midazolam, quinidine, and verapamil.

25. The method of claim 23, wherein the CYP1A2 substrate is phenacetin.

26. The method of claim 23, wherein the CYP2C9 substrate is selected from the group consisting of diclofenac and tolbutamide.

27. The method of claim 23, wherein the CYP2D6 substrate is selected from the group consisting of bufuralol, imipramine, and dextromethorphan.

28. The method of claim 23, wherein the CYP2C19 substrate is selected from the group consisting of omeprazole and S-mephenytoin.

29. The method of claim 23, wherein the CYP2E1 substrate is chlorzoxazone.

30. The method of claim 21, wherein the cytochrome P450 comprises a human cytochrome P450.

31. The method of claim 30, wherein the human cytochrome P450 is selected from the group consisting of CYP1A2, CYP2B6, CYP2C8, CYP2C9, CYP2C19, CYP2D6, CYP2E1, CYP3A4, CYP3A5, CYP3A7, and combinations thereof.

32. The method of claim 31, wherein the human cytochrome P450 is CYP3A4.

33. The method of claim 21, wherein the chemical species produced in the side reaction by the primary metabolic activity of the enzyme comprises a reactive oxygen species.

34. The method of claim 21, wherein the indicator compound precursor is selected from the group consisting of a fluorogenic compound, a colorimetric compound, a chemiluminescent compound, and combinations thereof.

35. The method of claim 34, wherein the indicator compound precursor is a fluorogenic compound.

36. The method of claim 21, wherein steps (a) through (c) are carried out in at least one well of a multi-well plate.

37. The method of claim 21, wherein the pre-set amount is selected from the group consisting of 25%, 33%, 50%, 67%, 75%, and 90%.

38. The method of claim 21, further comprising determining inhibitory potencies of a plurality of candidate compounds simultaneously.

39. The method of claim 38, wherein steps (a) through (c) are carried out in multiple wells of a multi-well plate.

40. The method of claim 21, further comprising quantifying a rate at which the indicator compound is produced in the presence of the candidate compound.
